(19) United States
(12) Patent Application Publication
(10) Pub. No.: US 2025/0058077 A1
(43) Pub. Date: Feb. 20, 2025

(54) AUDIO INFUSION SYSTEM AND METHOD

(71) Applicant: APPLIED INSIGHTS, LLC, RENO, NV (US)

(72) Inventors: MARK BRADFORD EVENSTAD, RENO, NV (US); WILLIAM MATTHEW CURLEY, FORT COLLINS, CO (US); JASON STUART DOESCHER, EDINA, MN (US); LEIGH MURRAY SMITH, RENO, NV (US)

(21) Appl. No.: 18/938,695

(22) Filed: Nov. 6, 2024

Related U.S. Application Data
(63) Continuation-in-part of application No. 18/105,373, filed on Feb. 3, 2023, now Pat. No. 12,165,622.

(51) Int. Cl. A61M 21/02; A61M 21/00; G06F 3/16; G10H 1/00; H04S 3/00; H04S 7/00

(52) U.S. Cl. CPC A61M 21/02; G06F 3/165; G10H 1/0025; H04S 3/008; H04S 7/307; A61M 2021/0027; G10H 2210/066; G10H 2210/111; G10H 2210/325; H04S 2400/01; H04S 2400/11; H04S 2400/13

(57) ABSTRACT

An audio infusion system and method are disclosed. A source audio track is separated into a plurality of audio tracks (e.g., instrumental, vocal, or mixes thereof) and the audio tracks are individually processed to generate a plurality of binaural beat tracks. At least one spatialized track is also generated by filtering the source audio track to provide a filtered track, generating one or more spatialization trajectories based on certain audio feature(s) of the source audio track (e.g., tempo) and a target end-state effect, and spatializing the filtered track using the spatialization trajectories. Other tracks may also be generated, such as one or more infrasonic tracks, ultrasonic tracks, enhanced bass tracks, and/or subharmonic tracks. The tracks may be played simultaneously or mixed for delivery to an end user device.

### CROSS-REFERENCE TO RELATED APPLICATIONS

This application is a continuation-in-part of and claims priority to U.S. patent application Ser. No. 18/105,373 filed on Feb. 3, 2023, which is incorporated herein by reference in its entirety.

### FIELD OF THE INVENTION

The present invention relates generally to audio systems and methods and, more particularly, to systems and methods for digitally processing audio recordings to provide a measurable enhanced effect for end users.

### DESCRIPTION OF RELATED ART

Different types of sounds may be used to entrain the human brain to reach a certain mental state. For example, when two tones having slightly different frequencies are played in the left and right ears simultaneously, the human brain perceives the creation of a third tone having a frequency equivalent to the difference between the two tones being played. This auditory illusion is called a binaural beat and, depending on its frequency, may be used to reduce stress and anxiety, increase focus and concentration, improve sleep quality, or provide other benefits. In some cases, an individual may choose to listen to binaural beats with music in order to improve the listening experience. However, the binaural beats are simply played as background to the music (or vice-versa)—i.e., the music material itself is not used for purposes of brainwave entrainment. Thus, there remains a need in the art for an improved method of delivering sounds with neuropsychological impact that overcomes drawbacks associated with existing methods and/or that offers other advantages compared to existing methods.

### SUMMARY OF THE INVENTION

The present invention is directed to methods and systems for infusing sounds into source audio tracks without disruption in the musical quality or perceived composition in order to provide desired psychological, neurological and/or physiological outcomes.

In one embodiment, a source audio track is separated into a plurality of audio tracks, such as multiple music stems, and each of the separated audio tracks is individually processed to generate a binaural beat track. Each binaural beat track is generated from an audio track by (a) transcribing the audio track to provide a transcription that includes an estimated fundamental frequency and/or an estimated amplitude envelope for each of a plurality of individual notes and (b) using the transcription to generate the binaural beat track. This embodiment provides multiple binaural beat tracks—one for each of the separated audio tracks—which provides multiple layers of stimulation. Because the binaural beat tracks are derived from the separated audio tracks, the perception of certain instruments and/or vocals in the audio tracks may be enhanced. Further, the delivery of multilayered binaural beats that are dynamically matched to the source audio track results in a polyphonic binaural beat experience that implements frequencies compatible with the tonal center of the source audio track without distorting the acoustic experience.

While existing methods of binaural beat implementation typically involve the delivery of one drone tone frequency in each of the left and right speaker channels, drawing from a limited frequency spectrum, the binaural beat generation method of the present invention draws from a series of pitches that are harmonically and melodically compatible with the source audio track. Since it is an established hypothesis that individuals respond to certain binaural “carrier” and “offset” frequencies with greater success than other frequencies on a subjective level, this method increases the probability of successful brainwave entrainment via the greater number and variety of source frequencies creating the binaural beat effect.

In another embodiment, the source audio track is processed to generate one or more spatialized tracks. Each spatialized track is generated from one or more spatialization trajectories that are derived from certain audio feature(s) of the source audio track (e.g., tempo, musical event density, harmonic mode, loudness, percussiveness, etc.) and a target end-state effect. Each spatialized track is configured so that the sound is perceived to move in time with the music—e.g., each spatialized track provides tempo-based spatialization. Spatialization creates sustained attention by directing the focus to a moving sound that enhances the neurological effect. Furthermore, tracking a moving sound activates spatial awareness.

In another embodiment, the source audio track is processed to generate both binaural beat tracks and one or more spatialized tracks, as described above. Additional tracks may also be generated, such as one or more infrasonic tracks, ultrasonic tracks, enhanced bass tracks, and/or subharmonic tracks. The various tracks may be played simultaneously or mixed for delivery of a single enhanced audio track to an end user device. It should be noted that the binaural beat stimulation and tempo-based spatialization are additive to the infrasonic and ultrasonic track synthesis and enhanced bass and subharmonic processing techniques so as to provide synergies in driving neurological responses.

Various embodiments of the present invention are described in detail below, or will be apparent to one skilled in the art based on the disclosure provided herein, or may be learned from the practice of the invention. It should be understood that the above summary of the invention is not intended to identify key features or essential components of the embodiments of the present invention, nor is it intended to be used as an aid in determining the scope of the claimed subject matter as set forth below.

### BRIEF DESCRIPTION OF FIGURES

Various exemplary embodiments of the present invention are described in detail below with reference to the attached drawing figures, wherein:

FIG. 1 is a block diagram of one embodiment of an audio infusion system that may be used to create enhanced audio tracks in accordance with the present invention;

FIG. 2 is a block diagram of one embodiment of an audio delivery and feedback system that may be used to deliver the enhanced audio tracks created by the audio infusion system of FIG. 1 to end user devices and receive feedback from end user devices/sensor devices that is passed to the audio infusion system of FIG. 1;

FIG. 3 is a flow diagram of one embodiment of an audio infusion process that may be performed in accordance with the present invention;

FIG. 4 is an exemplary graph showing the filtering of outlier notes as performed during

the “note filtering” step shown in FIG. 3;

FIG. 5 is a flow diagram of an exemplary process for generating a spatialization track;

FIG. 6 depicts an exemplary spatialization trajectory generated by the trajectory planner of FIG. 5;

FIG. 7 is a flow diagram of an exemplary process for setting the gains of the audio tracks; and

FIGS. 8A-8K depict example audio tracks at various stages of the audio infusion process shown in FIG. 3, including: a source audio track (FIG. 8A); individual tracks after the “gain processing” steps, including four binaural beat tracks (FIGS. 8B-8E), an infrasonic track (FIG. 8F), an ultrasonic track (FIG. 8G), a subharmonic track (FIG. 8H), an enhanced bass track (FIG. 8I), and a spatialized track (FIG. 8J); and a final enhanced audio track (FIG. 8K).

### DETAILED DESCRIPTION

The present invention is directed to methods and systems for infusing sounds into source audio tracks in order to provide desired psychological, neurological and/or physiological outcomes. While the invention will be described in detail below with reference to various exemplary embodiments, it should be understood that the invention is not limited to the specific configurations or methodologies of these embodiments. In addition, although the exemplary embodiments are described as embodying several different inventive features, one skilled in the art will appreciate that any one of these features could be implemented without the others in accordance with the present invention.

In the present disclosure, references to “one embodiment,” “an embodiment,” “an exemplary embodiment,” or “embodiments” mean that the feature or features being described are included in at least one embodiment of the invention. Separate references to “one embodiment,” “an embodiment,” “an exemplary embodiment,” or “embodiments” in this disclosure do not necessarily refer to the same embodiment and are also not mutually exclusive unless so stated and/or except as will be readily apparent to one skilled in the art from the description. For example, a feature, structure, function, etc. described in one embodiment may also be included in other embodiments, but is not necessarily included. Thus, the present invention can include a variety of combinations and/or integrations of the embodiments described herein.

In general terms, the methods of the present invention include various digital signal processing steps that are performed to infuse sounds into a source audio track and thereby produce an enhanced audio track that is configured to achieve a desired psychological, neurological and/or physiological outcome.

The source audio track may comprise any type of audio recording that includes vocals, instrumental music, and/or other types of sounds—including one channel music tracks (mono), two-channel music tracks (stereo), or multiple-channel music tracks (surround sound, spatial audio, etc.). In some embodiments, the source audio track is a stereo music track that has been professionally mixed and mastered. Of course, the present invention is not limited to the enhancement of commercial audio recordings, i.e., any type of audio recording may be enhanced in accordance with the teachings described herein.

Various types of sounds may be infused into the source audio track, such as binaural beats, tempo-based spatialization, infrasonic signals, ultrasonic signals, enhanced bass, subharmonics, and the like. It should be understood that any combination of these sounds may be used in a particular implementation, including (a) binaural beats only, (b) tempo-based spatialization only, (c) binaural beats and tempo-based spatialization, (d) binaural beats and infrasonic and/or ultrasonic signals, (e) tempo-based spatialization and infrasonic and/or ultrasonic signals; (f) binaural beats, tempo-based spatialization, and infrasonic and/or ultrasonic signals, and (g) any of the foregoing combinations with enhanced bass and/or subharmonics. Of course, other sound combinations and additional sounds may also be used within the scope of the present invention.

Sounds may be infused into the source audio track in different ways. One infusion method involves processing the source audio track in such a manner as to produce one or more audio tracks that are derived from the source audio track. Examples of audio tracks that may be produced using this infusion method include binaural beat track(s), spatialized track(s), enhanced bass track(s) and/or subharmonic track(s). Another infusion method involves generating one or more synthetic audio tracks. Examples of audio tracks that may be produced using this infusion method include infrasonic track(s) and/or ultrasonic track(s). Of course, both of the above infusion methods may be used, and all of the audio tracks may optionally be mixed to provide a single enhanced audio track. Thus, the “infusion” of sound into a source audio track as used herein means processing the source audio track to produce one or more audio tracks that are derived from the source audio track and/or generating one or more synthetic audio tracks, and optionally mixing the audio tracks to provide a single enhanced audio track—all for the purpose of obtaining a desired psychological, neurological and/or physiological outcome.

An enhanced audio track with the infused sounds may be delivered to an end user device via any known delivery method, such as streaming or downloading of an audio file to an end user device. The enhanced audio track may comprise a mixed audio track that may be played through stereo headphones, earphones, earbuds, one or more speakers, and the like. Alternatively, the enhanced audio track may comprise individual audio tracks that may be played simultaneously through multiple speakers or multiple headphone drivers.

The methods of the present invention may be performed in conjunction with additional pre-processing steps that occur before the steps described above. For example, a healthcare professional or other operator may determine the configuration settings of an input configuration that is used to produce the infused sound. These settings may be chosen so that the enhanced audio track provides a desired psychological, neurological and/or physiological outcome and, in that respect, the settings will vary between different end users. As another example, the source audio track may be separated from other sound elements prior to the steps described above—e.g., a movie track may include music, speech, and/or sound effects, wherein the music is separated from the speech and sound effects for enhancement as described herein. Other pre-processing steps will be apparent to one skilled in the art.

The methods of the present invention may also be performed in conjunction with additional post-processing steps that occur after the steps described above. For example, the enhanced audio track may be delivered to an end user via a production speaker system that is used for health or medical management. The end user's response to the enhanced audio track may then be measured for effectiveness by a healthcare professional. Other post-processing steps will be apparent to one skilled in the art.

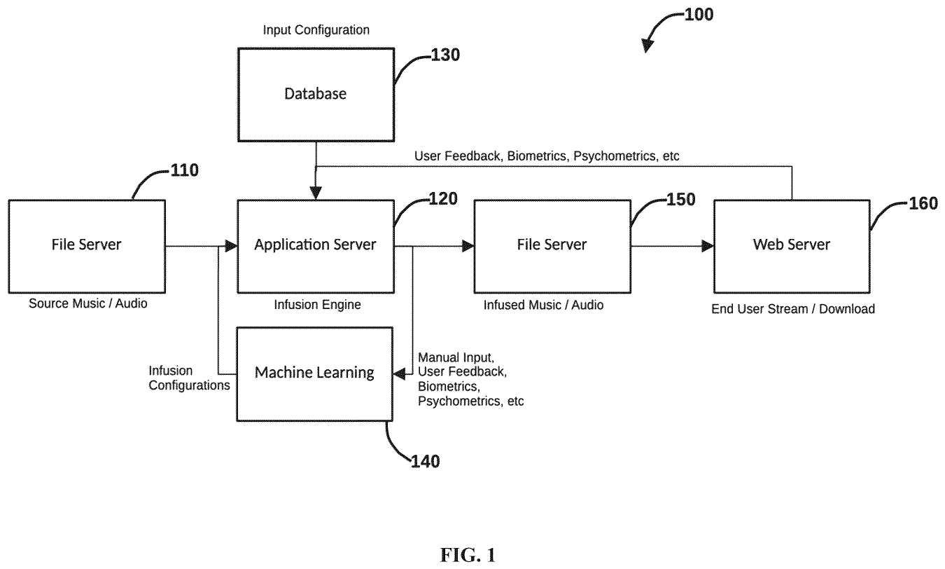

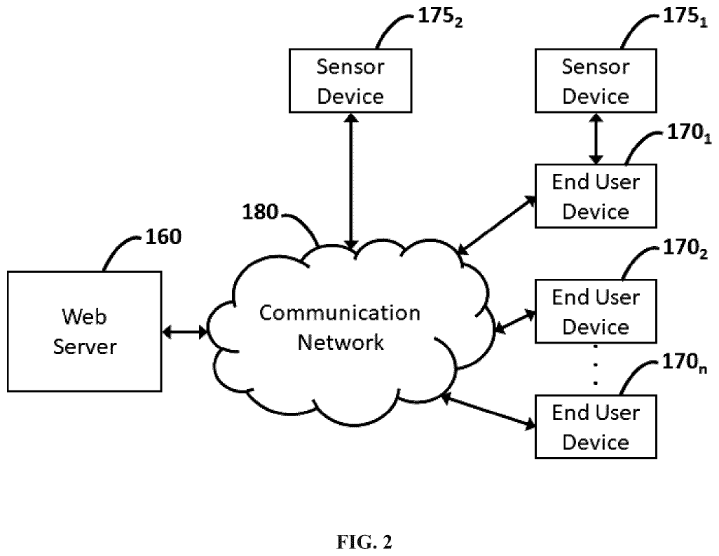

Referring to FIG. 1, one embodiment of an audio infusion system that may be used to create enhanced audio tracks in accordance with the present invention is shown generally as reference number 100. System 100 includes a file server 110 for storing source audio files, an application server 120 for executing a sound infusion application, a database server 130 for storing input configurations that have been manually entered by an operator, a machine learning server 140 for storing input configurations that have been generated through machine learning, a file server 150 for storing enhanced audio files, and a web server 160 for providing access to the enhanced audio files. In some embodiments, web server 160 also passes feedback data (e.g., user feedback, biometrics, psychometrics, demographic data, behavioral data, etc.) received from end user devices and/or sensor devices, as shown in FIG. 2, to the application server 120 for use in determining or modifying one or more configuration settings of an input configuration, as described below.

File server 110 may comprise any suitable computer hardware and software known in the art that is configured for the storage of source audio files that are available for enhancement by application server 120. The source audio files may be provided in any desired audio format. Examples of common audio formats include Advanced Audio Coding (AAC), Free Lossless Audio Codec (FLAC), Windows Media Video (WMV), Waveform Audio File Format (WAV), MPEG-1 Audio Layer 3 (MP3), and Pulse-Code Modulation (PCM). Of course, other audio formats known in the art may also be used.

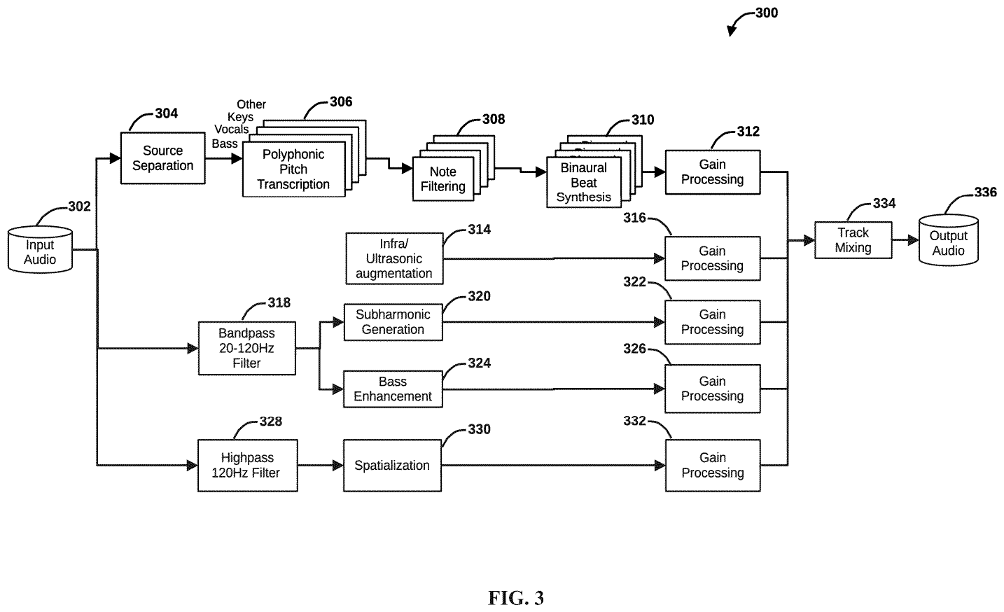

Application server 120 may comprise any suitable computer hardware and software known in the art that is configured to receive a source audio file from file server 110, receive the configuration settings of an input configuration from database server 130 and/or machine learning server 140, execute a sound infusion application to infuse sound into a source audio track in order to produce an enhanced audio track, and store an enhanced audio file on file server 150. In some embodiments, application server 120 may also receive feedback data from web server 160, as noted above, and provide the feedback data and optionally one or more manually entered configuration settings to machine learning server 140. As described below, the machine learning server 140 may use this information to determine or modify the configuration settings of the input configuration. The steps performed by the sound infusion application will be described in greater detail below in connection with FIGS. 3-7.

Database server 130 may comprise any suitable computer hardware and software known in the art that is configured to maintain a database for storing a plurality of input configurations that have been manually entered by an operator. The data elements for each input configuration may include a variety of different types of configuration settings, such as: (a) the frequency of binaural beats (in Hz); (b) the volume of binaural beats (in Loudness Units Full Scale (LUFS) units); (c) the frequency of infrasound (in Hz); (d) the volume of infrasound (in LUFS units); (e) the frequency of ultrasound (in Hz); (f) the volume of ultrasound (in LUFS units); (g) the subharmonic volume (in LUFS units); (h) the bass intensity (in LUFS units); (i) the bass volume (in LUFS units); (j) the volume of the spatialized audio content (in LUFS units); (k) the volume of the non-spatialized audio content (in LUFS units); (I) the frequency range of the spatialized audio content (in Hz); and/or (m) the frequency range of the non-spatialized audio content (in Hz). Of course, other configuration settings may also be stored in database 130 in accordance with the invention. In some embodiments, the operator is prompted to enter values for the configuration settings via a configuration file, although any data input method known in the art may be used.

Machine learning server 140 may comprise any suitable computer hardware and software known in the art that is configured to maintain and provide a set of machine learning models that are used to generate input configurations. It should be understood that any type of machine learning approach may be used, such as a recommendation engine that uses supervised learning, artificial neural networks, reinforcement learning, or other methods.

In general terms, the machine learning models provided by the machine learning server 140 are configured to accept one or more inputs and/or the infused output from application server 120 and adapt the configuration settings of an input configuration for optimal performance of the audio infusion application for each end user or end user group.

The inputs to the machine learning models provided by the machine learning server 140 may consist of a variety of different types of information, such as one or more of the following:
1. one or more manually entered configuration settings such as those stored in database server 130;
2. feedback data received from end user devices and/or sensor devices, as shown in FIG. 2, such as: (a) user feedback expressed as user preferences; (b) objective measurements such as biometrics (e.g., heart rate (HR), HR variability metrics, respiratory rate, body temperature, sleep metrics, and fitness related metrics (such as TRIMP)), psychometrics (e.g., use of clinically validated surveys or tools as a composite value metric, such as the Perceived Stress Scale (PSS) (S. Cohen 1994) or the Circumplex Model of Affect (J. Russell 1980)), and/or demographic data (e.g., age, sex, education level, and ethnicity); and/or (c) subjective measurements such as biometrics (e.g., use of self-reported metrics of physical state, such as Rate of Perceived Effort (RPE), sleep quality assessment, and comfort level), psychometrics (e.g., use of self-reported metrics of mood, emotion, and comfort level), and/or behavioral data (e.g., targeted goal state or ambition, scheduled metrics and action follow through);
3. a user's music playing behavior; and
4. music features, such as tempo, percussivity, event density, spectral measures such as centroid and spread, and learned features determined from deep learning of musical audio signals.

The configuration settings output by the machine learning server 140 may include one or more of the configuration settings specified above, such as: (a) the frequency of binaural beats (in Hz); (b) the volume of binaural beats (in Loudness Units Full Scale (LUFS) units); (c) the frequency of infrasound (in Hz); (d) the volume of infrasound (in LUFS units); (e) the frequency of ultrasound (in Hz); (f) the volume of ultrasound (in LUFS units); (g) the subharmonic volume (in LUFS units); (h) the bass intensity (in LUFS units); (i) the bass volume (in LUFS units); (j) the volume of the spatialized audio content (in LUFS units); (k) the volume of the non-spatialized audio content (in LUFS units); (I) the frequency range of the spatialized audio content (in Hz); and/or (m) the frequency range of the non-spatialized audio content (in Hz). Of course, other configuration settings may also be output by the machine learning server 140 in accordance with the invention.

Thus, it should be understood that the configuration settings for a particular end user or end user group may be manually input by an operator, may be determined through machine learning, or may be a combination of both. The recommended values for these configuration settings may vary between different end users or end user groups—i.e., the setting values recommended for one individual or group may be different than the setting values recommended for another individual or group. These setting values may be determined by experimentation based on various factors, such as the target end-state effect to be achieved, the intended duration of exposure to the enhanced audio track, whether a treatment plan requires the same or different stimuli to be delivered in different sessions, etc. Thus, it can be appreciated that the selection of the setting values enables the generation of an enhanced audio track that has been personalized or customized either manually or by a machine learning process for an end user or end user group.

File server 150 may comprise any suitable computer hardware and software known in the art that is configured for storage of the enhanced audio files generated by application server 120. The enhanced audio files may be provided in any desired audio format, such as AAC, FLAC, WMV, WAV, MP3, and PCM. Of course, other audio formats known in the art may also be used.

Web server 160 may comprise any suitable computer hardware and software known in the art that is configured to process requests to access (stream, download, etc.) the enhanced audio files stored on file server 150. As shown in FIG. 2, web server 160 may be used to deliver the enhanced audio files to a plurality of end user devices (including end user devices 170₁-170ₙ) via a communication network 180. Web server 160 may communicate with the end user devices via Hypertext Transfer Protocol (HTTP) (e.g., HTTP/1.0, HTTP/1.1 plus, HTTP/2, or HTTP/3), Hypertext Transfer Protocol Secure (HTTPS), or any other network protocol used to distribute data and web content. In some embodiments, web server 160 is also used to receive feedback data (e.g., user feedback, biometrics, psychometrics, demographic data, behavioral data, etc.) from end user devices (including end user devices 170₁-170ₙ) and/or sensor devices (including sensor devices 175₁ and 175₂.).

The end user devices may each comprise a mobile phone, a personal computing tablet, a personal computer, a laptop computer, or any other type of personal computing device that is capable of communication with web server 160 via communication network 180. In this embodiment, each of the end user devices utilizes an Internet-enabled application (e.g., a web browser or installed application) to communicate with web server 160. It should be understood that the end user devices may be used to play the enhanced audio tracks through stereo headphones, earphones, earbuds, one or more speakers, and the like. Alternatively, the end user devices may be used to store the enhanced audio tracks in memory (e.g., file copy, thumb drive, etc.) for later playback through another device (e.g., MP3 player, iPod®, etc.). In some embodiments, the end user devices are also used to input feedback data that is passed to application server 120 via web server 160.

The sensor devices may each comprise any device known in the art that is capable of collecting biometrics or other feedback data from an end user. In some cases, a sensor device is capable of direct communication with an end user device or is integrated into an end user device, wherein the end user device passes the feedback data to web server 160 via communication network 180. For example, in FIG. 2, sensor device 175₁ is in direct communication with end user device 170₁. In other cases, a sensor device is capable of communication with web server 160 via communication network 180, such as sensor device 175₂ in FIG. 2. Various arrangements of end user devices and sensor devices will be apparent to one skilled in the art.

Communication network 180 may comprise any network or combination of networks capable of facilitating the exchange of data between web server 160 and end user devices and/or sensor devices. In some embodiments, communication network 180 enables communication in accordance with one or more cellular standards, such as the Long-Term Evolution (LTE) standard, the Universal Mobile Telecommunications System (UMTS) standard, and the like. In other embodiments, communication network 180 enables communication in accordance with the IEEE 802.3 protocol (e.g., Ethernet) and/or the IEEE 802.11 protocol (e.g., Wi-Fi). Of course, other types of networks may also be used within the scope of the present invention.

It should be understood that system 100 is an exemplary embodiment and that other embodiments may not include all of the servers shown in FIG. 1 or may include additional servers that are not shown in FIG. 1. In particular, the system may be implemented with any number and combination of application servers, database servers, file servers, web servers, and the like, which are either co-located in the same geographic location or located in different geographic locations and connected to each other via communication network 180. Thus, the system may be implemented with any number and combination of servers that are either co-located or geographically dispersed.

Further, in another embodiment, the sound infusion process that is used to create enhanced audio tracks in accordance with the present invention is performed on a single computing device—e.g., a mobile phone, a personal computing tablet, a personal computer, a laptop computer, or any other type of personal computing device. In this case, the various servers of the system 100 shown in FIG. 1 would not be required. Thus, it can be appreciated that the computer-implemented methods of the present invention may be performed using any type of computing devices known in the art—including any one or combinations of servers, personal computing devices, and the like.

Referring to FIG. 3, one embodiment of a sound infusion process that may be performed to create enhanced audio tracks in accordance with the present invention is shown generally as reference number 300. This process may be performed, for example, by application server 120 via the execution of the sound infusion application noted above or by a personal computing device.

In step 302, a source audio track is provided as a digital audio track having one or multiple channels (e.g., mono, stereo, or surround sound). In some embodiments, a compressed or uncompressed audio file (e.g., an audio file provided in an AAC, FLAC, WMV, WAV, MP3, or PCM audio format) is read and decoded into a two-channel digital audio track. For example, application server 120 may read and decode an audio file retrieved from file server 110 to provide the two-channel digital audio track. In other embodiments, analog audio signals are passed through an analog to digital converter to provide the digital audio track.

In step 304, the source audio track is separated into a plurality of audio tracks, such as multiple stem tracks. Each stem track may comprise a vocals track or an instrumental track, which may include either one instrument or a combination of individual instruments that have been originally mixed together to produce the stem track. In the exemplary embodiment shown in FIG. 3, the source audio track is separated into four non-drum stem tracks—a vocals track, a bass track, a keyboards track, and an “other” pitched instruments track. Of course, the number of stem tracks extracted from the source audio track will vary between implementations, and may include, for example, two stem tracks (vocals and instrumental) or five stem tracks (vocals, drums, bass, keyboards, and other pitched instruments). Other stem models are also possible and may be used within the scope of the present invention. The source audio track may be separated into multiple stem tracks using any music source separation tool known in the art, such as Spleeter available from Deezer of Paris, France. In other embodiments, the source audio track is provided as a plurality of individual stem tracks, in which case the audio separation performed in step 304 would not be required.

Once the source audio track is provided as multiple audio tracks, subsequent processing of each of the audio tracks—including transcription (step 306), note filtering (step 308), and binaural beat synthesis (step 310)—can be performed in parallel on a multi-processor computer or across multiple computers. Steps 306, 308 and 310 will be described below in relation to the processing of one audio track; however, it should be understood that these steps will be performed in connection with each of the audio tracks that that are separated in step 304 or provided as individual audio tracks in step 302.

In step 306, each audio track is transcribed to provide information on each of the various individual notes contained in the audio track. As described below, the transcription may include, for example, an estimated fundamental frequency and/or an estimated amplitude envelope for each individual note in the audio track. If the source audio track comprises two or more channels (e.g., a stereo music track), the channels will be mixed to mono during the transcription process.

One skilled in the art will appreciate that this mixing may be performed in different ways, depending on the transcription tool used to perform the transcription. In some cases, the channels are mixed to mono and then a transcription of the single mixed channel is generated. In other cases, each channel is transcribed and then the channel transcriptions are combined. Of course, the channels of the source audio track may alternatively be mixed to mono prior to source separation in step 304, although this approach is generally not as efficient as using the transcription tool to perform the mixing during the transcription process of step 306.

Depending on the source, either monophonic or polyphonic pitch transcription may be used to estimate the fundamental frequency of each individual note in the audio track—e.g., monophonic pitch transcription may be used for inherently monophonic sources and polyphonic pitch transcription may be used for inherently polyphonic sources. For example, in the exemplary embodiment shown in FIG. 3, the vocals and bass stem tracks are monophonically transcribed, while the keyboards and “other” pitched instruments stem tracks are polyphonically transcribed.

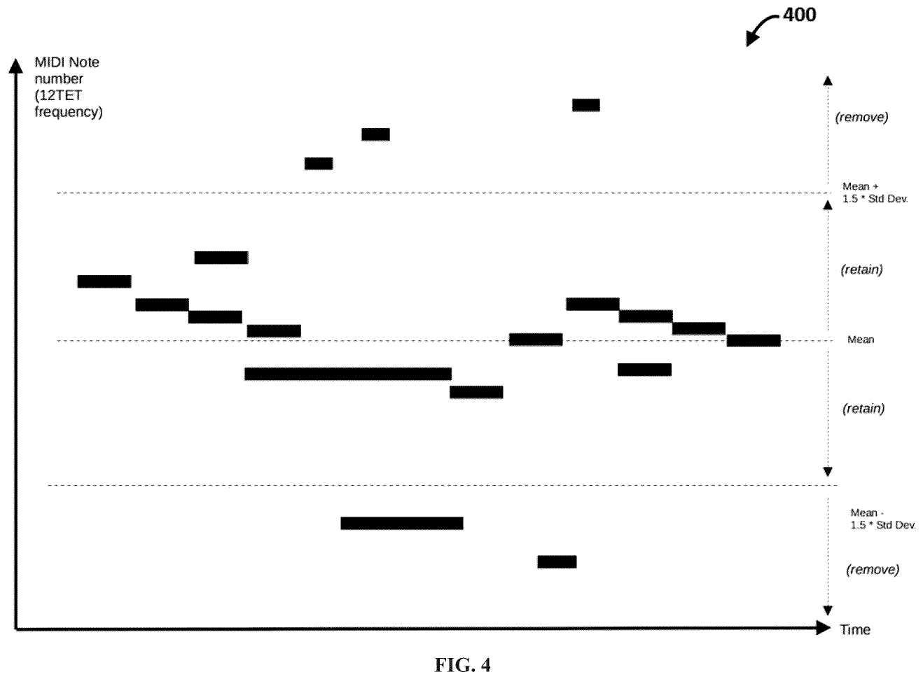

The pitch transcription may be represented in different formats. For example, the pitch transcription may be in the form of a musical note event list, such as a standard Musical Instrument Digital Interface (MIDI) file. In this case, each individual note is represented as a MIDI note number (i.e., a pitch quantized into a 12 tone equal-tempered note number) in relation to the time position of such note. An example of a MIDI file is shown in FIG. 4, which will be described below in connection with the note filtering process of step 308. As another example, the pitch transcription may be in the form of a time-frequency matrix (i.e. a spectrogram). In both examples, an estimate of the fundamental frequency of each individual note detected in the stem track is provided.

The transcription may also include an estimated amplitude envelope for each individual note in the audio track. As known in the art, an amplitude envelope for an individual note may be defined by four parameters: an attack time, a decay time, a sustain level, and a release time. During the transcription process, estimated values for these four parameters may be obtained for each individual note in the audio track. Alternatively, it is possible to use a fixed amplitude in which a single amplitude value averaged across time is provided in the settings of the input configuration. In this case, the transcription will only include frequency information-not amplitude information.

Each audio track may be transcribed using any transcription tool known in the art, such as the Melodyne transcription tool available from Celemony Software GmbH of Munich, Germany. Of course, it should be understood that other transcription tools may also be used in accordance with the present invention.

In step 308, the transcribed notes for each audio track are filtered in order to remove any outlier notes caused by errors in the pitch transcription process of step 306. One or more different note filtering methods may be used in accordance with the present invention.

One note filtering method involves identifying the outlier notes based on a statistical analysis of the pitch transcription for the stem track. Specifically, the pitch transcription is analyzed to calculate the mean and standard deviation of the estimated fundamental frequencies of the transcribed notes. Then, the outlier notes are identified as those having an estimated fundamental frequency that is either greater than an upper frequency limit or less than a lower frequency limit. The upper frequency limit has a value comprising the mean plus a first multiplier of the standard deviation, and the lower frequency limit has a value comprising the mean minus a second multiplier of the standard deviation. If the pitch transcription is represented as a MIDI file, the outlier notes are deleted from the musical note event list. On the other hand, if the pitch transcription is represented as a time-frequency matrix, the energy of the outlier notes is set to zero.

The first and second multipliers used to determine the upper and lower frequency limits, respectively, may be the same or different. FIG. 4 shows an example MIDI file in which the first and second multipliers are both set to 1.5. In this case, the individual notes having an estimated fundamental frequency greater than the upper frequency value (i.e., mean+1.5×standard deviation) or lower than the lower frequency value (i.e., mean−1.5×standard deviation) are removed from the musical event note list. Of course, the first and second multipliers may be different in other implementations. Further, the first and second multipliers may be the same for all of the audio tracks or may vary between different audio tracks.

In some embodiments, the first and second multipliers are determined by experimentation using multiple datasets of music selected to represent those typical for processing. Typically, the first and second multipliers will have a value in the range of 1.25 (in cases where more filtering is required) to 2.5 (in cases where less filtering is required). In most cases, the first and second multipliers are pre-set and do not need to be adjusted when processing different audio tracks. Of course, in other embodiments, the first and second multipliers may be part of the input configuration and, as such, would be entered by an operator or determined through machine learning.

Another note filtering method involves estimating the musical key of the audio track and then filtering out all transcribed notes with a fundamental frequency falling outside of that musical key. The musical key may be estimated using any key estimation algorithm known in the art, such as those described in C. L. Krumhansl and E. J. Kessler, Tracing the Dynamic Changes in Perceived Tonal Organization in a Spatial Representation of Musical Keys, Psychological Review, 89(4):334-368, 1982.

For example, if the musical key of a stem track is estimated to be A major and the pitch transcription process of step 306 generates a note list of (A3, C3, C #3, D3, E3, F3, G #3), the note filtering process of step 308 may filter that note list to (A3, C #3, D3, E3, G #3). In this case, certain notes are filtered out so that the remaining notes match the tonal center of the stem track—e.g., the A3, C #3, D3, E3, and G #3 pitches are compatible with the musical key of A major, while the C3 and F3 pitches are not compatible with the musical key of A major.

The musical key estimation and filtering method may be used by itself or in combination with the statistical analysis method described above. Of course, other note filtering methods known in the art may also be used within the scope of the present invention.

It should be understood that the note filtering methods described above may not be required if the errors in the pitch transcription process of step 306 are sufficiently low so as to fall within acceptable limits. In this regard, step 308 is optional and may not be performed in all implementations.

In step 310, the filtered transcription of each audio track is used to synthesize a corresponding binaural beat track. The binaural beat track is comprised of first and second binaural channels, each of which includes one or more sinusoidal signals that are derived from the transcribed notes in the filtered transcription. Different methods may be used to generate these sinusoidal signals.

In the exemplary embodiment shown in FIG. 3, the time-varying frequencies of the sinusoidal signals provided in the first and second binaural channels are determined by identifying a binaural beat frequency parameter, and then using this parameter in combination with the estimated fundamental frequency and temporal duration of each of the individual notes in the filtered transcription to determine such frequencies.

The binaural beat frequency parameter is included in the input configuration described above (i.e., the frequency of binaural beats (in Hz)), and is representative of a magnitude of frequency deviation from the estimated fundamental frequency of each transcribed note. Specifically, the binaural beat frequency parameter is added to the estimated fundamental frequency to determine the corresponding frequency of a sinusoidal signal provided in the first binaural channel and, conversely, the binaural beat frequency parameter is subtracted from the estimated fundamental frequency to determine the corresponding frequency of a sinusoidal signal provided in the second binaural channel. For example, if the binaural beat frequency parameter is 2 Hz and the estimated fundamental frequency of a transcribed note is 440 Hz, then the corresponding frequencies of the sinusoidal signals provided on the first and second binaural channels are 438 Hz and 442 Hz, respectively. This same approach is used for all transcribed notes. Thus, the binaural effect is synthesized by a low frequency difference between the sinusoidal signals provided on the first and second binaural channels.

For stems that are processed polyphonically, the filtered transcription may include multiple transcribed notes that occur at the same time—e.g., the “other” pitched instruments stem track may include notes associated with each of two or more instruments. The estimated fundamental frequencies of these transcribed notes are used to synthesize multiple (i.e., simultaneously sounding) sinusoidal signals. For stems that are processed monophonically, only one sinusoidal signal will occur at a time. Thus, it can be appreciated that each of the first and second binaural tracks may include one to multiple sinusoidal signals within the scope of the present invention.

In the exemplary embodiment shown in FIG. 3, the amplitude of the sinusoidal signals provided in the first and second binaural channels may be determined in different ways. In some embodiments, the estimated amplitude envelopes of the notes in the filtered transcription are used to control the relative amplitudes of the sinusoidal signals that are synthesized. For example, if the transcribed amplitude varies over time, then the amplitude of the binaural sinusoidal signals will also vary over time. In other embodiments, a binary beat volume parameter included in the input configuration described above (which defines a single amplitude value averaged across time) is used to determine the amplitude of the binaural sinusoidal signals. It can be appreciated that this approach provides a simplified way to determine the amplitude of the sinusoidal signals that does not require transcription of the estimated amplitude envelopes of the individual notes.

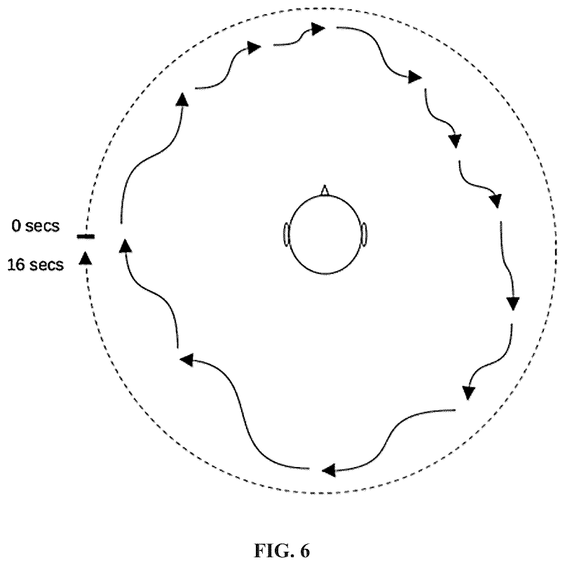

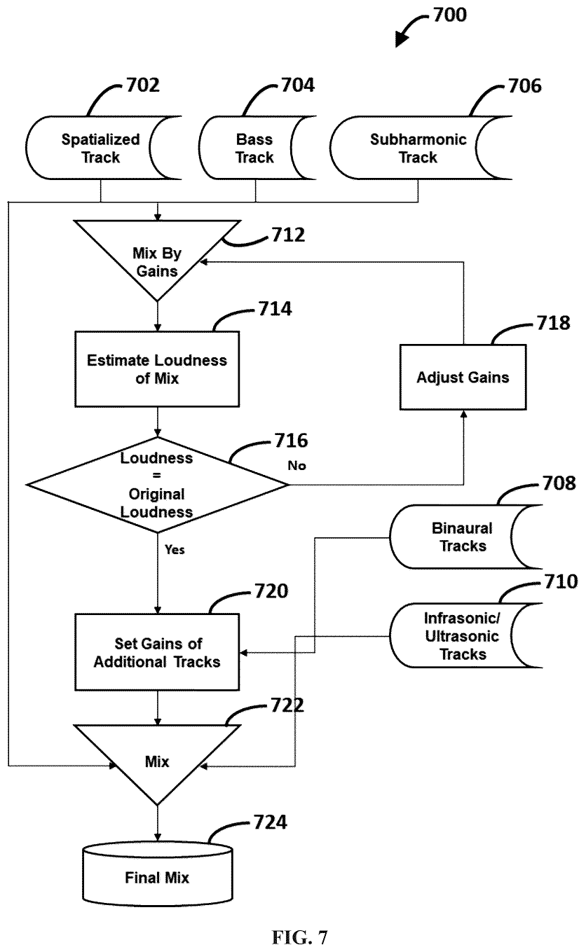

In step 312, the gains of the binaural beat tracks (i.e., one binaural beat track corresponding to each audio track) are adjusted using the gain setting process described below in connection with FIG. 7. Alternatively, any gain adjustment method known in the art, including use of a fixed gain, may be used to set the gains of the binaural beat tracks.

In step 314, one or more infrasonic tracks and/or one or more ultrasonic tracks are synthesized, each of which comprises a sinusoidal signal having a frequency and an amplitude that are fixed across the track. In some embodiments, the infrasonic and/or ultrasonic tracks are not derived from the source audio track (unlike the other tracks described herein). In other embodiments, the infrasonic and/or ultrasonic tracks are derived from the source audio track. For example, the amplitude envelope of the source audio track may be used to amplitude modulate the infrasonic and/or ultrasonic tracks.

For each infrasonic track, the frequency of the sinusoidal signal is determined from an infrasound frequency parameter included in the input configuration described above (i.e., the frequency of infrasound (in Hz)) and the amplitude of the sinusoidal signal is determined from an infrasound volume parameter included in the input configuration described above (i.e., the volume of infrasound (in LUFS units)). Typically, the frequency of the infrasound has a value in the range of 0.25 Hz to 20 Hz. The selected frequency is chosen to be complementary to the frequencies of the source audio track, either by manual selection, or by use of machine learning. The frequency chosen for each infrasonic track can be selected as complementary by combined reference to the neurological and physiological sensitivity of the listener to probe frequencies, and by reference to subharmonics of identified prominent frequencies in the source audio track. The balance between these contributions is determined either by manual selection, or by use of machine learning.

For each ultrasonic track, the frequency of the sinusoidal signal is determined from an ultrasound frequency parameter included in the input configuration described above (i.e., the frequency of ultrasound (in Hz)) and the amplitude of the sinusoidal signal is determined from an ultrasound volume parameter included in the input configuration described above (i.e., the volume of ultrasound (in LUFS units)). Typically, the frequency of the ultrasound has a value in the range of 20 kHz to the Nyquist frequency (one-half the audio sample rate). The selected frequency is chosen to be complementary to the frequencies of the source audio track, either by manual selection, or by use of machine learning. The frequency chosen for each ultrasonic track can be selected as complementary by combined reference to the neurological and physiological sensitivity of the listener to probe frequencies, and by reference to high frequency harmonics of identified prominent frequencies in the source audio track. The balance between these contributions is determined either by manual selection, or by use of machine learning.

It should be understood that the number of infrasonic tracks and/or ultrasonic tracks will vary between different contexts—i.e., some implementations will synthesize one or more infrasonic tracks, some implementations will synthesize one or more ultrasonic tracks, and some implementations will synthesize a combination of one or more infrasonic tracks and one or more ultrasonic tracks. It can be appreciated that the input configuration will include a frequency parameter and a volume parameter for each of these tracks.

In step 316, the gains of the one or more infrasonic tracks and/or the one or more ultrasonic tracks are adjusted using the gain setting process described below in connection with FIG. 7. Alternatively, any gain adjustment method known in the art, including use of a fixed gain, may be used to set the gains of the infrasonic and/or ultrasonic tracks.

In step 318, the source audio track is filtered to remove non-bass frequencies. In the exemplary embodiment shown in FIG. 3, a band pass filter having a passband of 20 Hz to 120 Hz is used to remove the non-bass frequencies. Of course, other methods may alternatively be used, e.g., source audio separation may be used to remove the non-bass instruments. Thus, the bass frequency content of the source audio track is provided for processing in steps 320 and 324.

In step 320, a subharmonic track is generated by synthesizing subharmonics of the bass frequency content of the music audio using a subharmonic generator. Any subharmonic generator known in the art may be used to create subharmonic frequencies below the fundamental frequencies of the bass frequency content, typically using harmonic subdivision methods. The subharmonic track may comprise non-sinusoidal signals derived from a combination of frequencies drawn from the source audio track using spectral analysis, such as a fast Fourier transform (FFT). The subharmonic volume (in LUFS units) provided in the input configuration described above is used to set an amplitude for the subharmonic track that is at a desired balance with the other musical elements within the final mixed track (e.g., not too overpowering in intensity, not inaudible, etc.).

In step 322, the gain of the subharmonic track is adjusted using the gain setting process described below in connection with FIG. 7. Alternatively, any gain adjustment method known in the art, including use of a fixed gain, may be used to set the gain of the subharmonic track.

In step 324, an enhanced bass track is generated by enhancing the bass frequency content of the music audio using the process described in U.S. Pat. No. 5,930,373, which is incorporated herein by reference. In this process, a bass intensity parameter included in the input configuration described above (i.e., the bass intensity (in LUFS units)) is used to determine the degree of bass enhancement of the processed track, while a bass volume parameter included in the input configuration described above (i.e., the bass volume (in LUFS units)) is used to determine the loudness of the processed track. One skilled in the art will appreciate that bass enhancements are desired for maximum effect (e.g., power, warmth, clarity, etc.).

In step 326, the gain of the enhanced bass track is adjusted using the gain setting process described below in connection with FIG. 7. Alternatively, any gain adjustment method known in the art, including use of a fixed gain, may be used to set the gain of the enhanced bass track.

In step 328, the source audio track is filtered to remove bass frequencies. In the exemplary embodiment shown in FIG. 3, a high pass filter is used to remove frequencies below 120 Hz. Of course, other filtering methods may alternatively be used. For example, source audio separation may be used to remove the bass instruments—in this case, source audio separation is used as a specialized form of filtering to remove the bass frequencies. A low pass filter may also be used to remove frequencies above 20 kHz in order to reduce high frequencies from sibilant sounds (e.g., cymbals) that may create audible artifacts from spatialization processing. Then, in step 330, a spatialized track is generated by spatializing the non-bass frequency content of the source audio track.

In other embodiments, a single audio track that has been separated from the source audio track, such as a particular stem track, is spatialized to generate a spatialized track. If the single audio track contains bass frequencies (e.g., a piano stem track), the single audio track may be filtered to remove the bass frequencies prior to spatialization. Thus, one skilled in the art will appreciate that steps 328 and 330 may be performed in connection with the entire source audio track (as in the exemplary embodiment) or a single source-separated audio track.

In step 332, the gain of the spatialized track is adjusted using the gain setting process described below in connection with FIG. 7. Alternatively, any gain adjustment method known in the art, including use of a fixed gain, may be used to set the gain of the spatialized track.

In step 334, the various audio tracks described above—i.e., binaural beat tracks, infrasonic and/or ultrasonic tracks, subharmonic track, enhanced bass track, and spatialized track—are mixed to produce an enhanced audio track. In the exemplary embodiment, the mixing is performed by summation and averaging to produce a two-channel digital audio track. Then, in step 336, the enhanced audio track is stored as a compressed or uncompressed audio file (e.g., an audio file provided in an AAC, FLAC, WMV, WAV, MP3, or PCM audio format). For example, with reference to FIG. 1, application server 120 may store the audio file in file server 150. The audio file may then be streamed or downloaded to an end user device for playback through stereo headphones, earphones, earbuds, one or more speakers, and the like. It is also possible to produce individual enhanced tracks (as opposed to a mixed track), wherein the individual enhanced tracks may be played simultaneously by the end user.

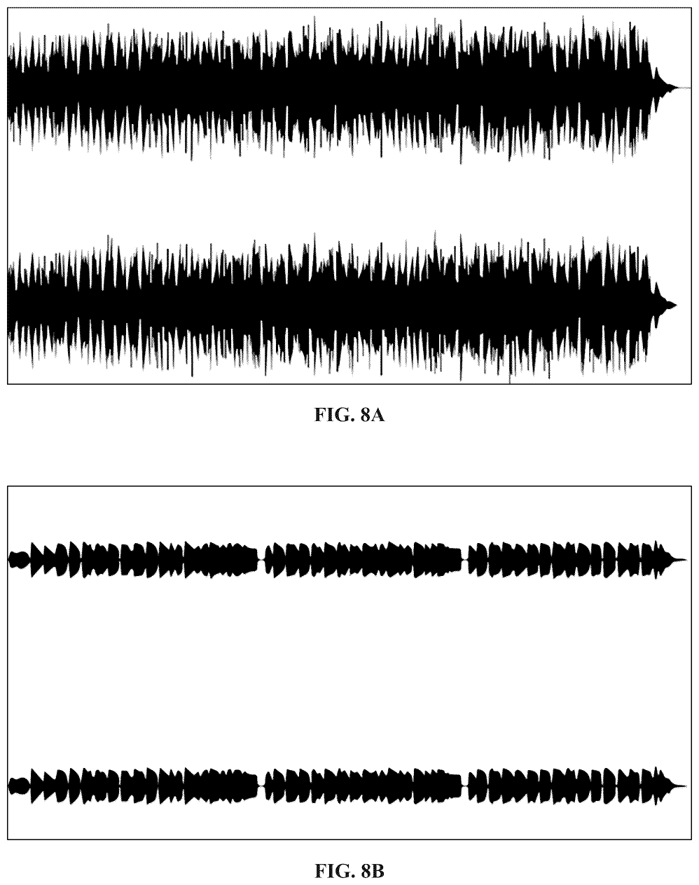

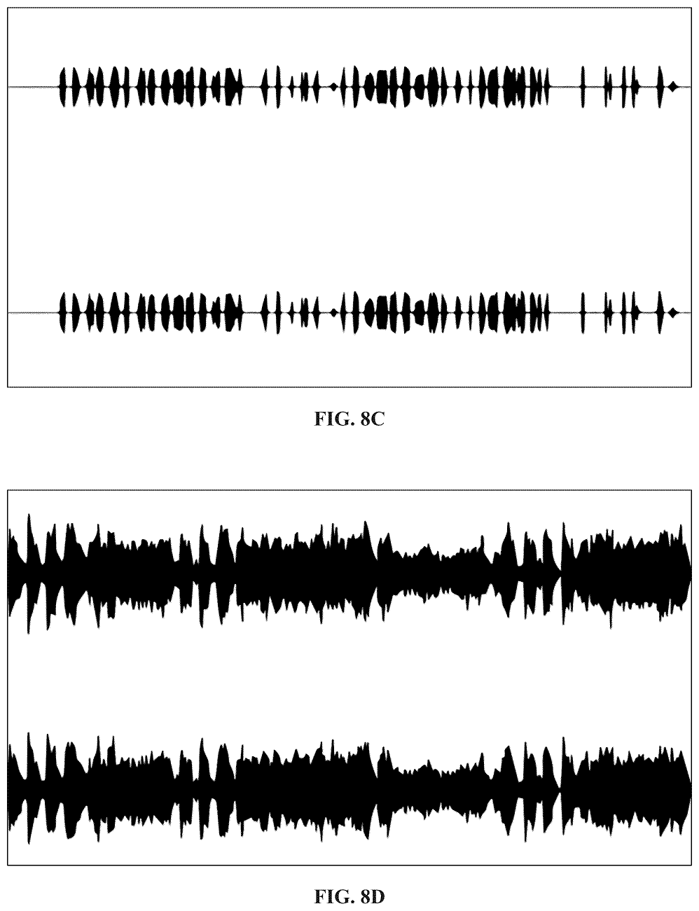

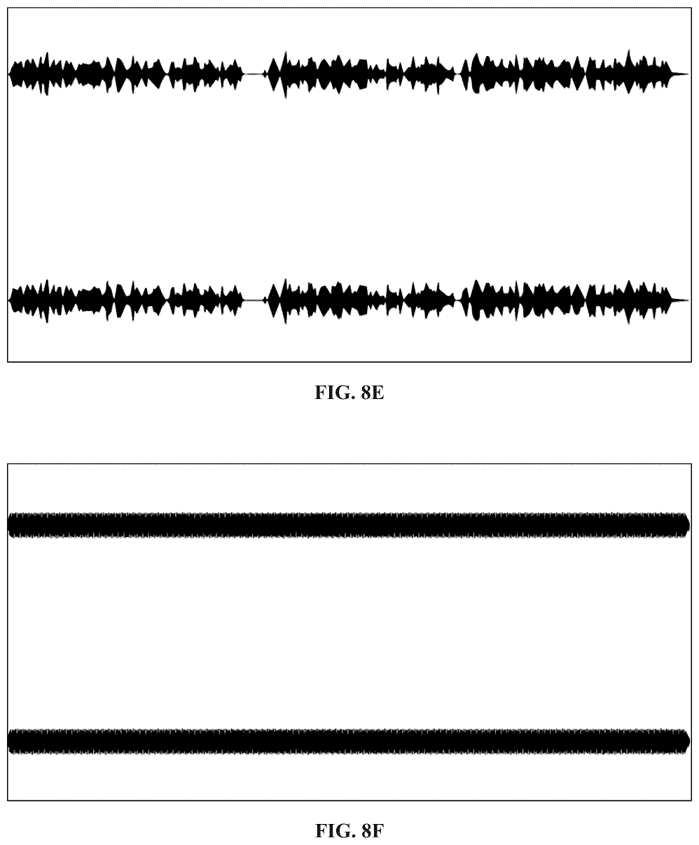

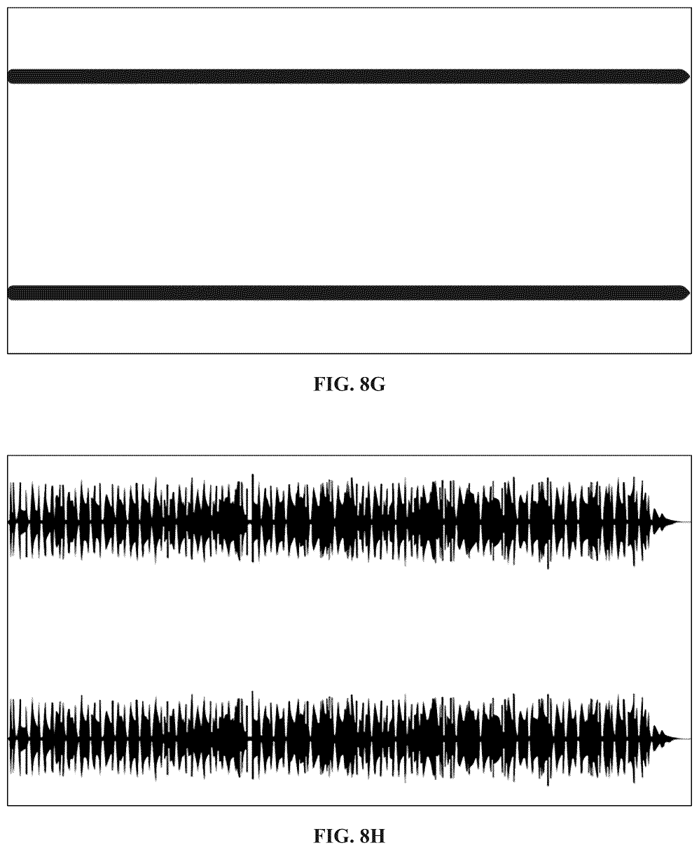

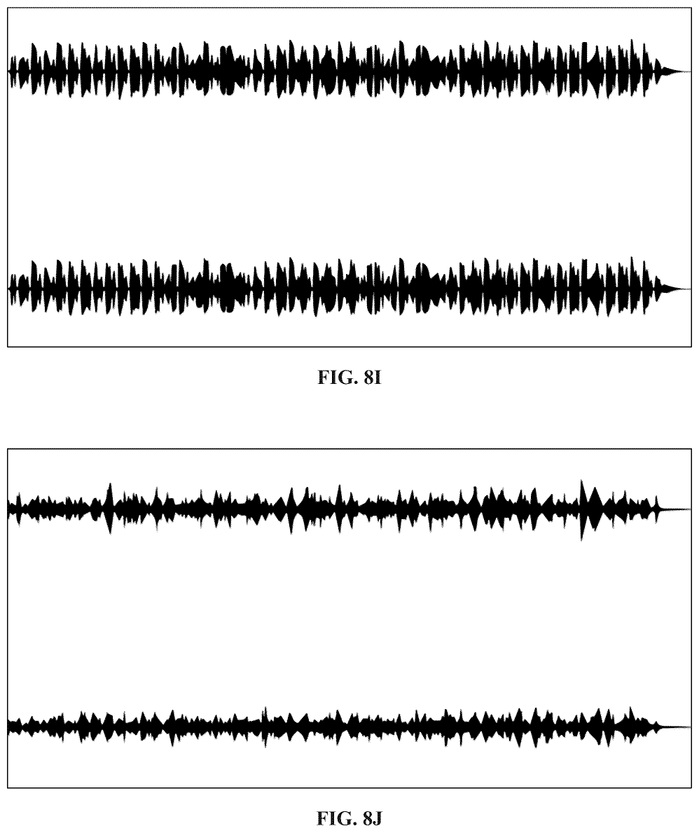

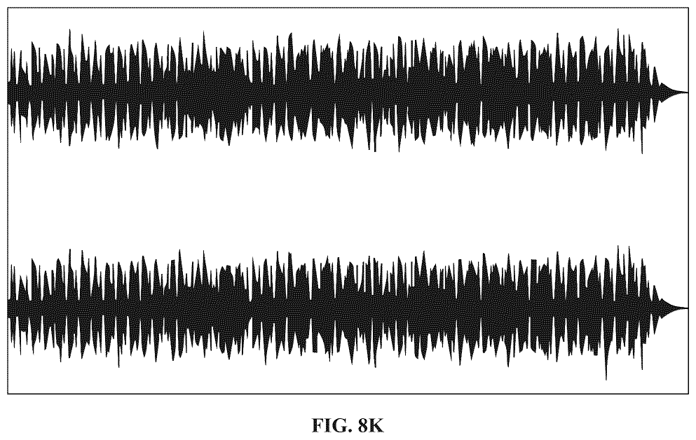

FIGS. 8A-8K depict example audio tracks at various stages of the sound infusion process shown in FIG. 3. Specifically, FIG. 8A depicts a source audio track obtained in step 302; FIG. 8B depicts a binaural beat track corresponding to the bass stem track after gain adjustment in step 312; FIG. 8C depicts a binaural beat track corresponding to the vocals stem track after gain adjustment in step 312; FIG. 8D depicts a binaural beat track corresponding to the keyboards stem track after gain adjustment in step 312; FIG. 8E depicts a binaural beat track corresponding to the “other” pitched instruments stem track after gain adjustment in step 312; FIG. 8F depicts an infrasonic track after gain adjustment in step 316; FIG. 8G depicts an ultrasonic track after gain adjustment in step 316; FIG. 8H depicts a subharmonic track after gain adjustment in step 322; FIG. 8I depicts an enhanced bass track after gain adjustment in step 326; FIG. 8J depicts a spatialized track after gain adjustment in step 332; and FIG. 8K depicts a final enhanced audio track after mixing in step 334.

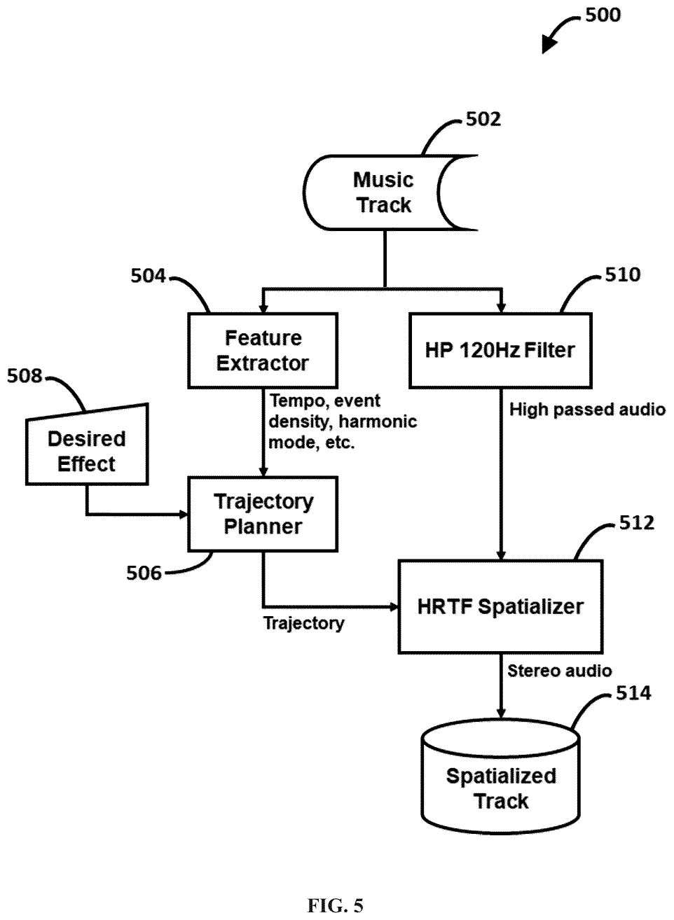

Referring to FIG. 5, one embodiment of a process for generating a spatialization track in accordance with the present invention is shown generally as reference number 500. This process may be performed, for example, as steps 328 and 330 of the method shown in FIG. 3.

In step 502, the source audio track is obtained. In step 504, one or more features are extracted from the source audio track, such as (a) a tempo of the source audio track (in beats per minute (BPM)), (b) a musical event density of the source audio track (in events per second), (c) a harmonic mode of the source audio track (major, minor, etc.), (d) a loudness of the source audio track (in LUFS units), and/or (e) a percussiveness of the source audio track (using leading edge transient detection). These features may be extracted using any feature extraction algorithms known in the art, and are then fed to a trajectory planner for processing in step 506, described below. Of course, other audio features may be extracted from the source audio track and fed to the trajectory planner within the scope of the present invention.

Examples of tempo estimation software that may be used to estimate the tempo of the source audio track include the Traktor software available from Native Instruments GmbH of

Berlin, Germany, the Cubase software available from Steinberg Media Technologies GmbH of Hamburg, Germany, the Ableton Live software available from Ableton AG of Berlin, Germany, and the Hornet Songkey MK4 software available from Hornet SRL of Italy. The tempo may also be estimated using the approaches described in D. Bogdanov et al., Essentia: An audio Analysis Library for Music Information Retrieval, 14th Conference of the International Society for Music Information Retrieval (ISMIR), pages 493-498, 2013.

The musical event density of the source audio track may be estimated using the approaches described in D. Bogdanov et al., Essentia: An audio Analysis Library for Music Information Retrieval, 14th Conference of the International Society for Music Information Retrieval (ISMIR), pages 493-498, 2013.

Examples of harmonic mode estimation software that may be used to estimate the harmonic mode of the source audio track include the Traktor software available from Native Instruments GmbH of Berlin, Germany, the Cubase software available from Steinberg Media Technologies GmbH of Hamburg, Germany, the Ableton Live software available from Ableton AG of Berlin, Germany, the Hornet Songkey MK4 software available from Hornet SRL of Italy, and the Pro Tools software available from Avid Technology, Inc. of Burlington, Massachusetts.

An example of a loudness estimation algorithm that may be used to estimate the loudness of the source audio track includes that specified in the European Broadcasting Union EBU-R128 specification for integrated loudness. Any software that is capable of producing loudness metering to the EBU-R128 standard is suitable for use in connection with the present invention.

The percussiveness of the source audio track may be estimated using the approaches described in F. Gouyon et al., Evaluating Rhythmic Descriptors for Musical Genre Classification, Proceedings of the AES 25th International Conference, pages 196-204, 2004.

In step 508, an end-state effect intended to be induced in the end user is obtained. Examples of end-state effects include, but are not limited to: (a) CALM (calm-grounded-controlled)—e.g., balanced, grounded presence, increased mental clarity and control, (b) MOTIVATE (motivated-inspired-energized)—e.g., increased energy and performance, elevated creative productivity and self-efficacy, (c) SLEEP (relaxed-tranquil-sleepy)—e.g., self-soothing, quieting the mind, transitioning to and maintaining a deeper sleep state, (d) CREATE (undistracted-meditative-focused)—e.g., purposeful attention, creative focus and productivity, (e) COMFORT (comforted-relieved-soothed)—e.g., directed focus to positive affect, attention to function and body signals, which may reduce discomfort, (f) RECOVER (recovered-restored)—e.g., improved restoration of body and mind, enhanced physiologic response to stress, and (g) PERFORM (driven-enhanced-in flow state—e.g., augmented attention, intensity, and duration of performance. Of course, other end-state effects may be used in accordance with the present invention. The end-state effect is then fed to the trajectory planner for processing in step 506, described below.

In step 506, the trajectory planner generates a spatialization trajectory that is derived from the audio feature(s) extracted from the source audio track and the target end-state effect. The spatialization trajectory may be a two-dimensional (2D) trajectory or a three-dimensional (3D) trajectory, depending on the implementation and desired effect. In particular, a 2D trajectory enables control of the perceived distance and lateral excursion of a sound; in a 3D trajectory, it is also possible to control the vertical excursion of the sound. Typically, multiple trajectory loops are generated for a source audio track—e.g., a trajectory loop for each musical section of the source audio track—to create the overall spatialization trajectory. The trajectory loops may comprise periodic trajectory loops (such as the trajectory loop shown in FIG. 6, discussed below) or non-periodic trajectory loops. The trajectory loops may all have the same trajectory parameters, or, the trajectory parameters may vary between different trajectory loops. A number of different approaches may be used to generate the spatialization trajectory.

In some embodiments, the audio feature(s) extracted from the source audio track and the target end-state effect are used to index into a preset trajectory loop—i.e., a series of control points that define the trajectory loop. Examples of four preset trajectory loops—each of which is associated with a tempo of the source audio track and a target end-state effect—are provided in Table 1 below:

TABLE 1

| Preset | | |
|---|---|---|
| Trajectory | | |
| Loop | Tempo | End-State Effect |
| #1 | 120-140 BPM | Effect A |
| #2 | 90-120 BPM | Effect A |
| #3 | Any | Effect B |
| #4 | 80-110 BPM | Effect C |

It should be understood that the preset trajectory loops provided in Table 1 are merely examples. Any number of preset trajectory loops may be used—each of which is associated with one or more audio features extracted from the source audio track and a target end-state effect. Once the control points of a trajectory loop are obtained, the tempo of the source audio track is used to render the distance between the control points to thereby create the trajectory loop.

In other embodiments, the spatialization trajectory is determined using a combination approach that includes lookup from a set of preset trajectory loops, as described above, and modulation of the control points of the selected trajectory loop using one or more of the audio feature(s) extracted from the source audio track. For example, the audio feature(s) extracted from the source audio track may be used to modify the amplitude of a trajectory loop (i.e., the degree of lateral and/or vertical excursion of a sound away from the listener's position), the frequency of a trajectory loop (i.e., the rate of path traversal), and/or other trajectory parameters. An example algorithm that uses this approach is provided below:
1. A tuple of tempo range category, musical genre category, and target end-state effect category are used to index into a table of trajectory loops, where a single trajectory loop is specified as a list of three dimensional Bezier spline control points.
2. The percussiveness parameter is bivalent normalized to the range from −1.0 to 1.0. It is then used as a scaling parameter to the azimuth of the trajectory loop, controlling the specific lateral excursion of the trajectory with respect to the listener's position.
3. The event density parameter is bivalent normalized to the range from −1.0 to 1.0. It is then used as a scaling parameter to the elevation of the trajectory loop, controlling the specific vertical excursion of the trajectory with respect to the listener's position.
4. The harmonic mode parameter is converted to a binary value—i.e., a binary value of “0” if the mode is one of the major modes (Ionian, Lydian, Mixolydian) or a binary value of “1” if the mode is one of the minor modes (Aeolian, Dorian, Phrygian, Locrian). If the binary value is “0”, the trajectory loop is not filtered. However, if the binary value is “1”, a low pass filter is applied to the trajectory loop, on each of the three dimensions, with the cutoff frequency scaled by the tempo parameter (measured in beats per minute), multiplied by the loudness parameter, in order to smooth the trajectory loop to a degree determined by features of the music.
5. The tempo parameter (measured in beats per minute) is used to compute the specific rate of the path traversal in seconds.

An example trajectory loop that may be generated using the above algorithm is shown in FIG. 6. As can be seen, the tempo extracted from the source audio track determines the rate of the trajectory loop, which is 16 seconds in this example. The remaining extracted audio features determine the control points of the trajectory loop in terms of degree of excursion (in this example, how much the sound moves in space relative to a circle), rate of change of the position of the music, complexity of the trajectory in terms of overlap, or repeated paths. Of course, it should be understood that other algorithms will result in a different trajectory loop than the example shown in FIG. 6. It should be understood that this process is repeated for each musical section of the source audio track to generate multiple trajectory loops for the spatialization trajectory. The spatialization trajectory generated in step 506 is then passed to a spatializer for further processing in step 512.

In step 510, the source audio track is filtered to remove bass frequencies. It should be understood that this step uses the same process described above in connection with step 328 of the method shown in FIG. 3. Then, in step 512, a spatializer is used to spatialize the non-bass frequency content of the source audio track based on the spatialization trajectory generated in step 506 to generate a spatialized track 514. In the exemplary embodiment, the spatializer uses a head-related transfer function (HRTF) spatialization algorithm, such as Dolby Atmos Personalized Rendering available from Dolby Laboratories, Inc. of San Francisco, California or dearVR Pro available from Dear Reality of Dusseldorf, Germany, in which each trajectory loop of the spatialization trajectory generated in step 506 is used to set the position of the sound within a virtual room. It should be noted that the position of the sound may be animated at a rate that is determined from the tempo of the source audio track. Thus, the sound is perceived to move in time with the music—i.e., the spatialized track provides tempo-based spatialization as the added sound.

It should be understood that the present invention is not limited to the generation of a spatialized track in accordance with the processes described above and that other processes may be used.

For example, in some embodiments, the trajectory planner is configured to generate a spatialization trajectory that is derived from the audio feature(s) extracted from the source audio track and the target end-state effect, as described above. However, instead of spatializing the entire source audio track, the spatializer is configured to use that spatialization trajectory to spatialize a single audio track that has been separated from the source audio track, such as a particular stem track, in order to generate a spatialized track. If the single audio track contains bass frequencies (e.g., a piano stem track), the single audio track may be filtered to remove the bass frequencies prior to spatialization.

In other embodiments, the trajectory planner is configured to generate a spatialization trajectory that is derived from the audio feature(s) extracted from a single audio track that has been separated from the source audio track, such as a particular stem track, and the target end-state effect. In one embodiment, the spatializer is configured to use that spatialization trajectory to spatialize the single audio track in order to generate a spatialized track. If the single audio track contains bass frequencies (e.g., a piano stem track), the single audio track may be filtered to remove the bass frequencies prior to spatialization. In another embodiment, the spatializer is configured to use that spatialization trajectory to spatialize the non-bass frequency content of the entire source audio track in order to generate a spatialized track.

In yet other embodiments, the trajectory planner is configured to generate a spatialization trajectory that is derived only from extracted audio feature(s)—e.g., either audio feature(s) extracted from the source audio track or audio feature(s) extracted from a single audio track that has been separated from the source audio track, such as a particular stem track. The spatialization trajectory is not derived from a target end-state effect in these embodiments. The spatializer is then configured to use that spatialization trajectory to spatialize either the non-bass frequency content of the entire source audio track or the single audio track (which may be filtered to remove bass frequencies) in order to generate a spatialized track.

In yet other embodiments, the trajectory planner is configured to generate a spatialization trajectory that is derived only from a target end-state effect. The spatialization trajectory is not derived from extracted audio feature(s) in these embodiments. The spatializer is then configured to use that spatialization trajectory to spatialize either the non-bass frequency content of the entire source audio track or a single audio track that has been separated from the source audio track, such as a particular stem track (which may be filtered to remove bass frequencies) in order to generate a spatialized track.

Further, in some embodiments, the process may use more than one filter in relation to the source audio track to provide multiple tracks that are spatialized simultaneously. For example, a first filter may remove frequencies below 120 Hz to provide a first track, a second filter may pass frequencies of 20 Hz to 120 Hz to provide a second track, a third filter may remove frequencies outside of 400 Hz to 1,000 Hz to provide a third track, and a fourth filter may remove frequencies outside of 1,000 Hz to 4,000 Hz to provide a fourth track. Such filtering could alternatively be in the form of source separation as described above. A different spatialization trajectory may be used to spatialize each of these tracks to thereby generate multiple spatialized tracks. Other examples will be apparent to one skilled in the art.

Referring to FIG. 7, one embodiment of a process for setting the gains of the audio tracks in accordance with the present invention is shown generally as reference number 700. This process may be performed, for example, in place of steps 312, 316,322, 326 and 332 of the method shown in FIG. 3. It will be seen that this process uses an iterative gain setting algorithm to set the gains of the audio tracks in order to ensure that mixing of the audio tracks does not result in an overdriven signal.

As an initial matter, the spatialized track is obtained in step 702, the enhanced bass track is obtained in step 704, the subharmonic track is obtained in step 706, the binaural beat tracks are obtained in step 708, and the infrasonic and/or ultrasonic tracks are obtained in step 710.

In step 712, the spatialized track, enhanced bass track, and subharmonic track are mixed by gains. In step 714, the loudness of the mix is estimated using one or more metrics, such as the loudness estimation algorithm conforming to the European Broadcasting Union standard EBU-R128 and true peak measures. In step 716, it is determined whether the metrics measured in step 714 match those of the source audio track, such as whether the loudness of the mix matches the loudness of the source audio track and whether the true peak measure matches that of the source audio track. Thus, multiple loudness metrics (e.g., integrated loudness and true peak) can be measured simultaneously to determine if multiple mixing goals are met. If not, in step 718, the gains of the spatialized track, enhanced bass track, and subharmonic track are iteratively adjusted until the loudness metrics substantially match the loudness metrics of the source audio track. Any multi-parameter estimation method, including machine learning, for example, stochastic gradient descent of multilayer perceptrons, may be used to determine the gain adjustment in each iteration. Of course, it is also possible to use more simplified methods, such as a hill-climbing estimate, to adjust the gains.

When the loudness metrics of the mix match the loudness metrics of the source audio track, in step 720, the final gain of the mix is used to set the gains of the binaural beat tracks and the infrasonic and/or ultrasonic tracks. In the exemplary embodiment, the gains of the binaural beat tracks and the infrasonic and/or ultrasonic tracks are determined by multiplying the final gain of the mix by a constant, i.e., a fixed loudness ratio that may be derived by experimentation with existing datasets or by data analysis. For example, if the final gain of the mix is −6 dB and the constant is 9, the gains of the binaural beat tracks and the infrasonic and/or ultrasonic tracks will be −54 dB.

In step 722, the various audio tracks—i.e., binaural beat tracks, infrasonic and/or ultrasonic tracks, subharmonic track, enhanced bass track, and spatialized track—are mixed to produce an enhanced audio track. Then, in step 724, the enhanced audio track is stored as an audio file. It should be understood that steps 722 and 724 use the same processes described above in connection with steps 334 and 336 of the method shown in FIG. 3.

The description set forth above provides several exemplary embodiments of the inventive subject matter. Although each exemplary embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The use of any and all examples or exemplary language (e.g., “such as”) provided with respect to certain embodiments is intended merely to better describe the invention and does not pose a limitation on the scope of the invention. Also, the use of relative relational terms, such as “first” and “second,” are used solely to distinguish one unit or action from another unit or action without necessarily requiring or implying any actual such relationship or order between such units or actions. No language in the description should be construed as indicating any non-claimed element essential to the practice of the invention.

The use of the terms “comprises,” “comprising,” or any other variation thereof, are intended to cover a non-exclusive inclusion, such that a process, method, or system that comprises a list of elements does not include only those elements, but may include other elements not expressly listed or inherent to such process, method, or system.

While the present invention has been described and illustrated hereinabove with reference to several exemplary embodiments, it should be understood that various modifications could be made to these embodiments without departing from the scope of the invention. Therefore, the present invention is not to be limited to the specific configurations or methodologies of the exemplary embodiments, except insofar as such limitations are included in the following claims.

What is claimed and desired to be secured by Letters Patent is as follows:

1. A computer-implemented method for generating one or both of a plurality of binaural beat tracks and a spatialized track that are customized for an end user, comprising:
determining a plurality of configuration settings for the end user;
using the configuration settings to generate one or both of the binaural beat tracks and the spatialized track; and
outputting the one or both of the binaural beat tracks and the spatialized track for delivery to the end user.

2. The computer-implemented method of claim 1, wherein each of the binaural beat tracks corresponds to one of a plurality of audio tracks separated from a source audio track, wherein each of the audio tracks comprises a plurality of individual notes, and wherein each of the binaural beat tracks is generated from one of the audio tracks by (a) transcribing the audio track to provide a pitch transcription that includes an estimated fundamental frequency for each of the individual notes in the audio track and (b) using the pitch transcription to control generation of the binaural beat track, wherein the binaural beat track comprises at least a first sinusoidal signal provided on a first binaural channel and at least a second sinusoidal signal provided on a second binaural channel.

3. The computer-implemented method of claim 1, wherein the spatialized track is generated by (a) identifying an audio track to be spatialized, wherein the audio track comprises one of a source audio track or an audio track separated from the source audio track, (b) generating a spatialization trajectory comprising a plurality of trajectory loops, wherein each of the trajectory loops is generated by indexing into a preset trajectory loop based on one or both of (i) one or more audio features extracted from the source audio track or the source-separated audio track and (ii) a target end-state effect intended to evoke in the end user one or more desired psychological, neurological or physiological outcomes, and (c) spatializing the audio track using the spatialization trajectory to generate the spatialized track.

4. The computer-implemented method of claim 1, wherein the configuration settings for a first end user are different than the configuration settings for a second end user.

5. The computer-implemented method of claim 1, wherein the configuration settings comprise one or more of (a) a frequency of binaural beats, (b) a volume of binaural beats, (c) a volume of spatialized audio content, (d) a volume of non-spatialized audio content, (e) a frequency range of spatialized audio content, and (f) a frequency range of non-spatialized audio content.

6. The computer-implemented method of claim 1, wherein one or more of the configuration settings are manually input by an operator.

7. The computer-implemented method of claim 1, wherein one or more of the configuration settings are determined via application of a machine learning model.

8. The computer-implemented method of claim 7, wherein the machine learning model has a plurality of inputs, wherein the inputs include one or both of (a) one or more manually entered configuration settings and (b) feedback data received from an end user device or a sensor device.

9. The computer-implemented method of claim 8, wherein the feedback data comprises one or more of user feedback, biometrics, psychometrics, demographic data, and behavioral data.

10. A computer-implemented method for generating binaural beat tracks, comprising:
generating a plurality of binaural beat tracks each of which corresponds to one of a plurality of audio tracks separated from a source audio track, wherein each of the audio tracks comprises a plurality of individual notes, and wherein each of the binaural beat tracks is generated from one of the audio tracks by (a) transcribing the audio track to provide a pitch transcription that includes an estimated fundamental frequency for each of the individual notes in the audio track and (b) using the pitch transcription to control generation of the binaural beat track, wherein the binaural beat track comprises at least a first sinusoidal signal provided on a first binaural channel and at least a second sinusoidal signal provided on a second binaural channel; and
outputting the binaural beat tracks.

11. The computer-implemented method of claim 10, wherein each of the audio tracks comprises one of an instrumental track, a mix of a plurality of instrumental tracks, a vocal track, or a mix of a plurality of vocal tracks.

12. The computer-implemented method of claim 10, wherein the pitch transcription comprises a monophonic pitch transcription.

13. The computer-implemented method of claim 10, wherein the pitch transcription comprises a polyphonic pitch transcription.

14. The computer-implemented method of claim 10, further comprising:
identifying a binaural beat frequency parameter representative of a magnitude of frequency deviation;
adding the binaural beat frequency parameter to the estimated fundamental frequency for each of the individual notes to determine a plurality of time-varying frequencies for the first sinusoidal signal provided on the first binaural channel; and
subtracting the binaural beat frequency parameter from the estimated fundamental frequency for each of the individual notes to determine a plurality of time-varying frequencies for the second sinusoidal signal provided on the second binaural channel.

15. The computer-implemented method of claim 10, further comprising:
identifying a binaural beat volume parameter; and
using the binaural beat volume parameter to control an amplitude of the first sinusoidal signal provided on the first binaural channel and the second sinusoidal signal provided on the second binaural channel.

16. The computer-implemented method of claim 10, wherein the pitch transcription includes an estimated amplitude envelope for each of the individual notes in the audio track, and wherein the method further comprises:
using the estimated amplitude envelope for each of the individual notes to control a plurality of time-varying amplitudes for the first sinusoidal signal provided on the first binaural channel and the second sinusoidal signal provided on the second binaural channel.

17. The computer-implemented method of claim 10, wherein the pitch transcription includes an estimated fundamental frequency for each of a plurality of outlier notes, and wherein the method further comprises:
filtering the outlier notes from the pitch transcription prior to generating the binaural beat track.

18. The computer-implemented method of claim 10, further comprising:
identifying an audio track to be spatialized, wherein the audio track comprises one of the source audio track or an audio track separated from the source audio track;
spatializing the audio track using a spatialization trajectory to generate a spatialized track; and
mixing the binaural beat tracks and the spatialized track to generate a mixed track.

19. A computer-implemented method for generating a spatialized track, comprising:
identifying an audio track to be spatialized, wherein the audio track comprises one of a source audio track or an audio track separated from the source audio track;
generating a spatialization trajectory comprising a plurality of trajectory loops, wherein each of the trajectory loops is generated by indexing into a preset trajectory loop based on one or both of (a) one or more audio features extracted from the source audio track or the source-separated audio track and (b) a target end-state effect intended to evoke in an end user one or more desired psychological, neurological or physiological outcomes;
spatializing the audio track using the spatialization trajectory to generate a spatialized track; and
outputting the spatialized track.

20. The computer-implemented method of claim 19, further comprising filtering the audio track to be spatialized prior to spatializing the audio track.

21. The computer-implemented method of claim 20, wherein filtering the audio track comprises applying a high pass filter to the audio track.

22. The computer-implemented method of claim 20, wherein filtering the audio track comprises using source audio separation to remove one or more bass instruments from the audio track.

23. The computer-implemented method of claim 19, wherein each of the trajectory loops corresponds to a musical section of the audio track.

24. The computer-implemented method of claim 19, wherein the preset trajectory loop comprises a plurality of control points.

25. The computer-implemented method of claim 24, wherein at least one of the audio features comprises a tempo that is used to render a distance between the control points of the preset trajectory loop.

26. The computer-implemented method of claim 24, wherein each of the trajectory loops is further generated by modulating the control points of the preset trajectory loop based on the one or more audio features.

27. The computer-implemented method of claim 19, wherein each of the audio features comprises one of a tempo, a musical event density, a harmonic mode, a loudness, and a percussiveness measurement.

28. The computer-implemented method of claim 19, wherein the target end-state effect comprises one of calm, motivate, sleep, create, comfort, recover, and perform.

29. The computer-implemented method of claim 19, further comprising:
synthesizing a plurality of binaural beat tracks each of which corresponds to one of a plurality of audio tracks separated from the source audio track; and
mixing the spatialized track and the binaural beat tracks to generate a mixed track.
